(19) United States
(12) Patent Application Publication
(10) Pub. No.: US 2004/0106869 A1
(43) Pub. Date: Jun. 3, 2004

(54) Ultrasound tracking device, system and method for intrabody guiding procedures

(75) Inventor: Ronnie Tepper, Herzliya (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Ron-Tech Medical Ltd.

(21) Appl. No.: 10/306,159

(22) Filed: Nov. 29, 2002

(51) Int. Cl. A61B 8/14

(52) U.S. Cl. 600/443; 600/458

(57) ABSTRACT

Apparatus for precision location of a tool such as a surgical tool within an obscured region such as an internal space of the human or animal body, the apparatus comprising: a planar scanning unit for scanning planes within said obscured region using an imaging scan, and a locator, associated with said tool and with said scanning unit, for determining a location of said tool, and for selecting a plane including said tool location. The apparatus allows the planar scan to follow the tool automatically and saves skill and effort on the part of the surgeon.

### FIELD AND BACKGROUND OF THE INVENTION

The present invention relates to an ultrasound tracking device, system and method and, more particularly, but not exclusively to a device that uses three dimensional ultrasound imaging techniques and invasive tools.

Three dimensional ultrasound scanning is known from a number of patent applications including EP 0 920 642, 3D Ultrasound Recording Device, assigned to Synthes A G of Chur, Switzerland. An effective system for displaying 3D ultrasound imaging data is disclosed in U.S. Pat. No. 5,682,895 to Ishiguro.

Three-dimensional ultrasound has the advantage of acquiring a data set of images that can be accumulated to volume from a single ultrasonic window. The two dimensional slice data from the scanner or probe is used as input data for the three dimensional reconstructions.

Basics and Principles:

Three-dimensional imaging is based on two-dimensional imaging. Two dimensional imaging involves acquiring planar sweeps, and the three dimensional image is built up from a series of planes making up a volume. The data can be acquired by parallel, rolling or sweep type probe movements. The volume may then be displayed in various ways. In fact, three dimensional images can be produced using a number of different methods, the most complex of which comprises generating three dimensional images based on the acquisition of a large number of consecutive 2 dimensional images through the movement of the transducer.

The sweep acquisition can be done free-hand with or without orientation sensors or using a specialized transducer which sweeps a volume mechanically as a series of planes and allows the processing of these volumes in a standard manner. The volume is then digitally stored and can be displayed either as a multi-planar image showing three orthogonal planes or as a surface rendered image. The three perpendicular planes display the X, Y and Z axes with the understanding that the Z plane is one that can not be acquired directly.

An advantage of three-dimensional imaging is that it enables reorienting of the display plane after the volume has been acquired, allowing the other two planes to be viewed. In fact, further than that, it is possible to view a standard two-dimensional cross sectional image in any plane within the volume, and gives free access to viewing angles that are in fact inaccessible. The user is thus enabled to effectively rescan a patient by reviewing the saved volume in any two-dimensional plane, even if different from the original scan plane. Such an effective rescan is particularly useful, for example in fetal imaging where frequently a fetus being imaged is not in an ideal position. The acquired volume can be manipulated to display the image in a non-scanning reconstructed plane. The image of a fetal profile, for example, is necessary to image the chin, and may not be obtainable on a fetus that is not positioned properly. The volume of the fetal face can be displayed in any desired orientation, including a sagittal view, to optimize the fetal profile or any other area if interest. Returning to the question of representing the gathered volume data, the spatial orientation of sonogram sweep data is monitored throughout the process of acquisition and the data are then stored in the computer memory as a volume set. The relative position and orientation of the 2 dimensional images can be established using mechanical, electromagnetic or acoustic techniques. There are then three ways to evaluate the volume dataset:

1. Section reconstruction

2. Surface rendering.

3. Volume rendering.

By using three dimensional ultrasound there is the possibility to reconstruct and display any arbitrary chosen 2 Dimension section plane within the scanned area.

The volume scan may automatically be performed by a tilt movement added on top of the standard 2D scan mechanism.

Applications of 3D

There are many useful applications for three-dimensional volume acquisition of two-dimensional ultrasound. The first includes networking and the ability to send packets of information from one site to another. The information obtained with three-dimensional volume acquisition is far superior to simply a video or cine-loop of two dimensional information. Several studies have demonstrated the benefit of using three-dimensional volume sets to send to a remote location via electronic networks to a specialist, who may then review the data and render an interpretation. The specialist can interactively reorient the volume even if the imaging was not done in an ideal plane or if the fetus was not in a desired position. This enables sites distant from a central location to optimize their backup capabilities using remote “expert” consultation.

There is a definitely a learning curve to the ability to obtain a good volume set and the training of the sonographer or the physician obtaining the volumes as well as those reading it must be different than the training in standard 2D imaging. Training must include standard acquisitions of volumes that will display with a minimum of artifact. Sonographers must recognize inadequate volumes that cannot be used due to motion or other artifacts. The training of physicians reviewing the volumes must also include learning how to evaluate anatomy in orientations different from the original acquisition plane. A standardized protocol must also be in place so that the volumes are not viewed haphazardly but in a standard fashion and in multiple planes.

Further research evaluates the feasibility of performing a virtual patient examination using three-dimensional ultrasound acquired in one location and sent to remote locations to be read. They demonstrate that overall, 3D ultrasound could be used with diagnostic quality results comparable to standard two-dimensional ultrasound, although the reconstructed 3D image quality itself is generally lower than the directly acquired two-dimensional image. There are also differences among reviewers interpretations thus emphasizing the need for a standardization of acquisition and reviewing protocols for users. Three-dimensional virtual examination techniques are regarded as being particularly well suited for echocardiography, where volumes can be sent via the Internet to a tertiary fetal cardiology center to reevaluate a cardiac data set. Studies show that a three-dimensional virtual examination of the fetal heart is possible although there are still limitations that need to be worked out. Certainly the main cardiac connections can be viewed and reconstructed in different ways. This may be helpful to patients being scanned in remote locations where questions about cardiac anatomy may arise.

Ultrasound guided prostate seed brachytherapy is a new, non-invasive, outpatient procedure that uses 3D ultrasound imaging to assist with correct positioning of implants in order to treat patients in the early stage of prostate cancer.

The prostate brachytherapy procedure involves implanting tiny radioactive rice-sized pellets directly into the prostate, where they can irradiate the prostate from the inside. Physicians use ultrasound based three-dimensional imaging techniques to place seeds at exact spots and intervals so actual radiation only penetrates a short distance, thereby minimizing radiation to adjacent organs. The seeds emit radiation from inside the prostate for about nine months and then become inert, with no need for removal. Brachytherapy is effective for cancer that has not spread beyond the boundaries of the prostate gland.

The procedure is a three-step procedure; acquisition of two-dimensional images, processing and finally visualization or display. The data starts out as slices (images) taken at regular intervals using echo-endoscope, ultra-thin probes or laparoscopic probes using a computer controlled acquisition device which uses an algorithmic approach to obtain a three dimensional representation of the internal organ.

Image guided neurosurgery (IGNS) is a field that currently exists. However, it is generally felt that current techniques lack accuracy.

There are many aspects of an image-guided surgery system that can potentially introduce errors into the system. Since one of the objectives of IGNS is to achieve an accuracy and precision of better than 1 mm (particularly for functional stimulation, ablation and tissue implants), there is currently a great deal of effort being expended to identify sources of error and to propose means of eliminating them.

One currently used way of dealing with inaccuracy is to gather as accurate information as possible from the patient and to combine the data with that already gathered from an atlas of previously obtained data. Even the highest quality MR images fail to demonstrate some of the fine detail and structures necessary for the surgeon to perform certain procedures, for example, thalamotomy and pallidotomy. On the other hand, detailed atlases of these structures exist, and they may be merged, using a non-linear warping procedure, with the patient's MR images to provide additional guides and landmarks during surgery. These atlases may be complemented with a probabilistic electro-physiological atlas using data acquired at surgery.

Image-Guided Neurosurgery—Background

The central hypothesis of IGNS is that if the neurosurgeon can be provided with rich, image-based information describing the underlying anatomy, function, and vascularity, along with tools that allow him to interpret and use this information effectively, then surgical procedures can be made less invasive, and patient morbidity, hospitalization time and cost will be reduced.

These image-guided tools provide a virtual, non-invasive “window” into the body, allowing the surgeon visual access to anatomical details and physiological function that are not available using other means. Over the past 15 years, the work in this laboratory has evolved in parallel with both the explosion in computer power and capacity, and the development and refinement of 3-D diagnostic imaging modalities. It has taken maximum advantage of the increasing power and accuracy offered by technology, while at the same time ensuring that the tools are surgeon-friendly and cost effective.

The ultimate objective of minimally invasive neurosurgery is to remove completely the targeted lesion by damaging the smallest possible volume of brain tissue, causing the least trauma to the patient, to achieve the desired therapeutic result. To achieve this objective, the goal of an ideal IGNS system is to report the position of an intra-operative guidance device within the brain with perfect accuracy. This would require that the brain images presented to the surgeon on a video monitor always reflect the actual geometrical state of the brain. This goal is partly achieved by registering the image data to the patient by identifying common structures in the patient and the image. However, in reality, even if the patient-image registration problem has been addressed perfectly, most IGNS systems use preoperative image information and their accuracy is affected by many factors. Most can be attributed to failures of basic assumptions under the following three categories:

Image Assumptions: that the images used as guidance for surgery contain all of the relevant anatomical and functional information required for surgical guidance;

Instrumentation Assumptions: that the images are geometrically accurate, the tracking device is free of positioning error, the registration between the patient and image is correct, and the images are free from spatial distortion; and,

Brain Tissue Assumptions: that the equipment and volume of surgical interest form a completely rigid system, implying that the structures of interest within the brain remain in the same position during surgery with respect to the external fiducial points used for patient-image registration The following passage, describes the use of 3-D ultrasound in neurosurgery. “Recently, it was reported that current generation ultrasound imagers are capable of visualizing the intra-cranial vasculature, which can be reconstructed as 3-D volumes, and we have demonstrated the clinical applicability of combining intra-operative ultrasound images with pre-operative MRI. The next step is to use the target images to update the geometry of the pre-operative images. The goal may be achieved by developing strategies that match similar structures (vessels and tissue boundaries) that are detected in both ultrasound and MR images. This will allow inference of the displacement of a field of “tag-points” between the ultrasound and MR images. This displacement map will then be used to calculate the deformation necessary to match the pre-operative MRI to the ultrasound image. The original 3-D MRI data will thus be updated based on the changing morphology detected by ultrasound during surgery. Such fusion of MRI and 3-D ultrasound may result in a near real-time intra-operative imaging system that maintains the attributes of the pre-operative MRI. It will present substantial advantages over specialized intra-operative MR imaging systems, both in image resolution and cost.”

A paper by Aaron Fenster and Donal B. Downey of Imaging Research Laboratories, The J. P. Robarts Research Institute Ontario, Canada, discusses three-dimensional ultrasound imaging. According to the paper, ultrasonography, a widely used imaging modality for the diagnosis and staging of many diseases, is an important cost-effective technique, however, technical improvements are necessary to realize its full potential. 2D viewing of 3D anatomy, using conventional ultrasonography, limits our ability to quantify and visualize most diseases, causing, in part, the reported variability in diagnosis and ultrasound guided therapy and surgery. This occurs because conventional ultrasound images are 2D, yet the anatomy is 3D; hence, the diagnostician must integrate multiple images in his mind. This practice is inefficient, and may lead to operator variability and incorrect diagnoses. In addition, the 2D ultrasound image represents a single thin plane at some arbitrary angle in the body. It is difficult to localize and reproduce the image plane subsequently, making conventional ultrasonography unsatisfactory for follow-up studies and for monitoring therapy.

The authors have focused on overcoming these deficiencies by developing 3D ultrasound imaging techniques that can acquire B mode, color Doppler and power Doppler images. An inexpensive desktop computer is used to reconstruct the information in 3D, and then is also used for interactive viewing of the 3D images. They use 3D ultrasound images for the diagnosis of prostate cancer, carotid disease, breast cancer and liver disease and for applications in obstetrics and gynecology. In addition, they use 3D ultrasonography for image-guided minimally invasive therapeutic applications of the prostate such as cryotherapy and brachytherapy. Volume Measurements Another important clinical application of 3D is volume measurements calculations based on 3D volume acquisition. Only as a result of acquiring a complete volume is it feasible to make an accurate volume estimation for tissues or tissue regions under study. Real-Time 3D or 4D Researchers have attempted to use real-time 3D (otherwise known as 4D) in the assessment of fetal behavior during pregnancy. Although the number of frames per second is still less than required for a smooth real time image, there is enough information using continuous three-dimensional ultrasonographic images to display fetal activity. It is unclear however how much more information is available using 3D than 2D real-time, since fetal movement is readily visible in 2D, which have been used to study fetal movement successfully for many years. It may however be possible to image more of the fetal body at once using real-time 3D surface rendering than using the single slice standard 2D imaging. Multi-planar displays in real-time (3D) may also be advantageous to visualize movement in a non-scanning (or Z) plane. It remains to be seen whether real-time 3D (or 4D) sonography will play a role in the evaluation of fetal well-being during gestation.

Generally it requires a large amount of scanning and image processing to keep a standard one-dimensional ultrasound image updated for real time applications.

Considerably more processing is needed to keep a three dimensional ultrasound image updated for real time applications.

Uterine Procedures:

Moving now from fields where imaging is widely used, as of today intrauterine and cervical procedures are generally performed by an archaic “blind” technique, which is to say either the surgeon does not use imaging at all, or if he does use imaging then the tool he is using and the region he is operating on does not appear in the images or only appears with great effort. To name a few common procedures: Curettage or evacuation of the uterine cavity for diagnostic and/or therapeutic purpose including termination of pregnancy (TOP); Removal of an endometrial polyp or submucous myoma; Insertion or extraction of an intra-uterine contraceptive device; sampling of the endometrium and/or the endocervix for diagnostic purposes; embryo transfer during in-vitro fertilization (IVF); and tubal diagnostic for treatment procedures.

Due to lack of ability to image the tool, the above procedures are generally carried out blindly, relying on the surgeon's experience and “feel” through manual manipulation of the instruments over the uterus walls.

When the position or size of the uterus is incorrectly diagnosed or recognized by the surgeon, which is often the case with inexperienced physicians, uterine perforation may occur with remarkable ease. The chances of perforation are higher in the presence of cervical stances or uterine malignancy (endometrial or sarcoma). The dangers in such uterine perforation include bleeding and trauma to the abdominal viscera as well as damage to internal organs. Thus, hospitalization and exploration of the abdominal cavity by laparoscopy or laparotomy is often needed due to such accidental uterine perforation. Other possible unfortunate outcomes of such blind operation procedures include, for example, failure to completely remove uterine tissues such as placental or fetal tissues during termination of pregnancy, resulting in re-hospitalization (expensive) and the need for a second curettage under general anesthesia (high risk).

Currently the use of real-time monitoring and guiding of surgical procedures is very limited and usually performed only during complicated procedures (in many cases complications from unguided procedures). In such cases trans-abdominal probes are usually used but they have relatively limited resolution, they require keeping the patient's urinary bladder full during the operation, and they require additional operating staff.

Image-Guided Gynecologic Surgery—Background

There is a widely recognized need for, and it would be highly advantageous to have, an apparatus and method for real-time endovaginal sonographic guidance and monitoring of intra-uterine and cervical surgical and non-surgical procedures.

Such apparatus may enable the surgeon to perform such procedures safely, conveniently and efficiently. In particular, it would be advantageous because of substantially shortening the duration of the surgical procedures currently carried out under general anesthesia, and it would reduce the rate of complications associated with such procedures. Only ten years ago the amniocentesis procedure for pregnant women was performed without ultrasound or any other guidance means. The procedure was performed blindly, with the physicians feeling the position of and collecting the sample of the fluid whilst trying to avoid approaching the fetus. Today, no physician would attempt collection of amniotic fluid without the guidance of a real-time ultrasound image displaying the fetus and the applied needle, and any attempt at such a procedure without ultrasound guidance would be regarded as malpractice.

Abnormal uterine bleeding is a common reason for gynecological visits by women. Although many of these cases have a benign etiology, the possibility of malignancy must be ruled out. About 7% of postmenopausal women not receiving hormone replacement therapy (HRT), who present with uterine bleeding have a malignancy. Thus, postmenopausal bleeding is considered endometrial cancer until proven otherwise.

Patients who receive HRT for six months, and then present uterine bleeding, are generally recommended for undergo endometrial sampling.

Peri-menopausal women with abnormal bleeding are at increased risk of endometrial cancer secondary to their age and anovulatory cycles. Thus, all women with abnormal uterine bleeding in the peri-menopausal period require endometrial sampling.

Indications for endometrial biopsy in pre-menopausal women with abnormal bleeding are not as straightforward. Beyond adolescence, endometrial cancer should be considered in the differential diagnosis of abnormal uterine bleeding since up to 10% of women with endometrial carcinoma are diagnosed before the age of 45.

In women under the age of 40 with no risk factors, the chance of endometrial cancer is minimal. The most important risk factor in this group of women is irregular menstrual cycles, which is associated with a 14% chance of an abnormal endometrial biopsy, including benign and malignant lesions. Thus, an endometrial biopsy should be considered in almost all women with irregular cycles.

Other sub-groups who can be recommended to undergo endometrial sampling with biopsy include patients treated with Tamoxifen, and who then experience abnormal uterine bleeding. In postmenopausal women, the presence of any endometrial cells on a Pap smear is indicative of a need for endometrial sampling. In other women, the presence of atypical endometrial cells should warrant an endometrial biopsy. Patients with malignant endometrial cells on a Pap smear are at significant risk of endometrial cancer, often with high-grade malignancy.

Detecting the cause of abnormal uterine bleeding requires endometrial tissue sampling, which can be performed as an office procedure.

Previously, the gold standard for sampling the endometrium was dilatation and curettage (D&C) under general anesthesia. For full evaluation of bleeding, endocervical curettage should also be done to localize the source of bleeding. If no cause of bleeding can be found or if the tissue obtained is inadequate for diagnosis, D&C must be performed. It is now recognized that D&C is actually a “blind” sampling technique, which often samples less than half of the endometrium.

Another technique uses the endometrial Pipelle or Z-Sampler and has further simplified endometrial sampling. The Pipelle is a flexible polypropylene suction cannulas, generally having an outer diameter of 3.1 mm and its use is almost painless in most situations, with particular ease of use in the postmenopausal woman.

Although false negatives may occur in focal malignancy of the endometrium, it was found that the sensitivity and specificity of the Pipelle in endometrial tissue samplings compared with fractional curettage were 87.5 and 100 per cent, respectively. Guido et al also studied Pipelle biopsies in patients with known carcinoma undergoing hysterectomy. It was found that a Pipelle biopsy provided adequate tissue for analysis in 63 out of 65 patients (97%). Malignancy however, was detected in only 54 patients (83%). It was noted that tumors localized in a polyp or a small area of endometrium may go undetected. Guido et al concluded that the “Pipelle is excellent for detecting global processes in the endometrium.”

In yet another technique, Vabra, an aspirator is used to obtain tissue for histological examination. A narrow (3-4 mm) suction curette with a vacuum pump is used to perform curettage of an adequate endometrial sample, which allows histological diagnosis of hyperplasia and endometrial carcinoma. Rodriquez et al (26) studied hysterectomy specimens and showed that the percentage of endometrial surface sampled by the Pipelle biopsy was 4% versus 41% for the Vabra aspirator.

The accuracy of endometrial biopsy in detecting endometrial disease, especially cancer, is highly acceptable. In studies comparing endometrial biopsies to hysterectomy specimens, endometrial biopsy had sensitivities ranging from 83 to 96% for detecting endometrial cancer. Currently, endometrial biopsy has replaced D&C as the diagnostic test of choice for evaluation of abnormal bleeding as both tests have shown to be similarly accurate.

The above procedures comprise techniques for sampling endometrial tissue, which involve a certain risk of complication, attributed to the fact that they are performed without continuous visualization of the organs and operating tools during the procedure.

The use of endovaginal ultrasound in women at high risk of endometrial neoplasia is gaining popularity. Currently available evidence indicates that endovaginal ultrasonography is an acceptable alternative to endometrial biopsy as the initial step in evaluating abnormal vaginal bleeding in postmenopausal women from the standpoints of accuracy, patient acceptability, and cost. This indirect method of visualizing the uterine cavity and measuring endometrial thickness has a sensitivity of 96% for detection of endometrial cancer and 92% for detection of any endometrial disease (cancer, polyps, or atypical hyperplasia) in postmenopausal bleeding. The sensitivity of transvaginal ultrasound compares favorably with that of office endometrial biopsy; sensitivity estimates for biopsy published in the literature ranges from 85% to 95% (ref).

Nondirected office biopsy carried out alone without imaging suffers from a potential to miss the diagnosis of focal lesions such as polyps, submucous myomas, and focal hyperplasia in up to 18% of the patients.

Diagnostic hysteroscopy and sonohysterography are equally effective in assessing the endometrium. Krampl et al confirm the findings of others showing that ultrasound, hysterosonography, and diagnostic hysteroscopy are not sufficient to identify endometrial pathology. None of these diagnostic tests can in fact replace the use of biopsies for the diagnosis of endometrial abnormalities. Hysteroscopy and hysterosonography are useful in the diagnosis of focal intrauterine pathology, but in order to improve diagnostic accuracy they need to be combined with endometrial sampling. The problems outlined above indicate the need to have some form of coordination between imaging or like information gathering and tool operation. Such co-ordination is presently known from a system marketed as Safe-T-Choice™, which provides a combination solution for uterine sonography and intrauterine operative procedures via a technology which employs a transvaginal transducer that is connected to the instrument holding the cervix (tenaculum) via an adapter. The solution provides real time sonographic guidance for continuous viewing of the organs and tools during procedures performed within the uterine cavity.

The use of such a transducer can improve the outcome of intrauterine procedures, such as endometrial sampling, while exposing the patients to lesser risks. However, due to the planar nature of ultrasonic scanning, it requires effort on the part of the user to keep the necessary features in view.

The following references provide further background for this section and are hereby incorporated herein by reference.

Choo Y C, Mak K C, Hsu C, Wong T S, Ma H K. Postmenopausal uterine bleeding of nonorganic cause. Obstet Gynecol 1985; 66. 225-8.

Chambers J T, Chambers S K—Endometrial sampling: Who? Where? Why? With what? Clin Obstet Gynecol 1992; 35 (1) 28-39.

Brand A, Duduc-Lissoir J, Ehlen T G, Plante M. Diagnosis of endometrial cancer in women with abnormal vaginal bleeding. SOGC Clinical Practice Guidelines. J Soc Obstet Gynecol Can 2000; 22 (1): 102-4.

Udeff L, Langenberg P, Adashi E Y. Combined continuous hormone replacement therapy: a critical review. Obstet Gynecol 1995; 86: 306-16.

Apgar B S, Newkirk G R. Endometrial biopsy. Primary Care 1997; 24 (2): 303-26.

Bealy P S. Diseases of the uterus (Chapter 50). In: DanforthÕs Obstetrics & Gynecology, 8th Ed Lippincott, Williams & Wilkins, 1999: 846.

Brenton L A, Berman M L, Mortel R, et al. Reproductive, menstrual, and medical risk factors for endometrial cancer. results from a case-control study. Am J Obstet Gynecol 1992; 167: 1317-25.

Farrell S A, Samson S, Ash S, Flowerdew G, Andreou P. Risk categories for abnormal endometrial biopsy in dysfunctional uterine bleeding. J Soc Obstet Gynecol Can 2000; 22 (4): 265-9.

Dubeshtes B, Warshal D P, Angel C, et al. Endometrial carcinoma: the relevance of cervical cytology. Obstet Gynecol 1991; 77. 458-62

Stock R J, Kenbour L. A prehysterectomy curettage—Obstet Gynecol 1990; 76: 1000.

Fothergill D J, Brown V A, Hill A S. Histological sampling of the endometrium D a comparison between formal curettage and the Pipelle sampler. Br J Obstet Gynecol 1992, 99: 779-80.

Kaunitz A M, Masciello A, Ostrowski M, Rorvion E Z. Comparison of endometrial biopsy with the endometrial Pipelle and Vabra aspirator. J Reprod Med 1988; 33: 427.

Koss L G, Schreiber K, Oberlander S G, et al Detection of endometrial carcinoma and hyperplasia in asymptomatic women. Obstet Gynecol 1984; 64. 1-11

Stovall TG, Ling FW, Morgan PL A prospective, randomized comparison of the Pipelle endometrial sampling device with the Novak curette. Am J Obstet Gynecol 1991; 165: 1287-9.

Rodriquez G C, Yaqub N, King M E. A comparison of the Pipelle device and the Vabra aspirator as measured by endometrial denudation in hysterectomy specimens. Am J Obstet Gynecol 1993; 168: 55-9

Goldchmit R, Katz Z, Blickstein I, Caspi B, Dgani R. The accuracy of endometrial Pipelle sampling with and without sonographic measurement of endometrial thickness. Obstet Gynecol 1993; 82 (5): 727-30.

Kavak Z, Cayhan N, Pekin S. Combination of vaginal ultrasonography and Pipelle sampling in the diagnosis of endometrial disease. Aust NZ J Obstet Gynecol 1996; 36 (1): 63-6.

Stovall T G, Photopoulos G J, Poston W M, Ling F W, Sandles L G. Pipelle endometrial sampling in patients with known endometrial carcinoma. Obstet Gynecol 1991; 77 (6): 954-6.

Nand S L, Webster M A, Baber R, et al. Bleeding pattern and endometrial changes during continuous combined hormone replacement therapy. Obstet Gynecol. 1998; 91:678-684.

Dubinsky T J, Parvey H R, Gormaz G, et al. Transvaginal hysterosonography: comparison with biopsy in the evaluation of postmenopausal bleeding. J Ultrasound Med. 1995; 14:887-893.

Guido R S, Kanbour A, Ruhn M, et al. Pipelle endometrial sampling sensitivity in the detection of endometrial cancer. J Reprod Med. 1995; 40:553-555.

Stovall T G, Photopulos G J, Poston W M, et al. Pipelle endometrial sampling in patients with known endometrial carcinoma Obstet Gynecol. 1991; 77:954-956.

Smith-Bindman R, Kerlikowske K, Feldstein V A, et al. Endovaginal ultrasound to exclude endometrial cancer and other endometrial abnormalities: a meta-analytic review. JAMA 1998; 280:JMA80013.

Goldstein S R, Zeltser I I, Horan C K, et al Ultrasonography-based triage for perimenopausal patients with abnormal uterine bleeding. Am J Obstet Gynecol. 1997; 177:102-108.

Weber A M, Belinson J L, Bradley L D, Piedmonte M R. Vaginal ultrasonography versus endometrial biopsy in women with postmenopausal bleeding. Am J Obstet Gynecol. 1997; 177:924-929.

Steven R Goldstein, , Ilana Zeltser, B S, Camille K. Horan, R D M S, Jon R Snyder, , and Lisa B Schwartz, Ultrasonography-based triage for perimenopausal patients with abnormal uterine bleeding Am J Obstet Gynecol 1997; 177 102-8.

Rodriguez M H, Platt L D, Medearis A L, Lacarra M., Lobo R A. The use of transvaginal sonography for evaluation of postmenopausal size and morphology. Am J Obstet Gynecol 1998; 159:810-4.

Guido R S, Kanbour A, Ruhn M, Christopherson W A Pipelle endometrial sampling sensitivity in the detection of endometrial cancer. J Reprod Med 1995;40:553-5.

Annually some 100,000 millions of surgical procedures of the types discussed above are performed. The complication rate is between 3-6% for termination of pregnancy, and there is an associated inaccuracy rate of 10-20% for sampling specific targets. Most of these complications and inaccuracies occur in blind type procedures. Tracking systems for 3D ultrasound imaging are known, for example from International Patent Application WO 01/06924 to Bova et al. The application discloses a 3D ultrasound probe combined with a tracking device and an arrangement of probe position markers. The markers are tracked using infrared cameras and tracking data from the markers is used to provide a frame of reference to the ultrasound data. However, the frame of reference is absolute and fixed. There is no way of taking into account body movements, particularly breathing, pulse-related movements and other involuntary movements that may occur during surgery. There is no indication of how to relate the frame of reference to points of interest or indeed any way to recognize points of interest. Indeed scanning is limited to flat planes and if a surgical tool is being used, it difficult to ensure that the tool being used features in any of the planes being scanned.

U.S. Pat. No. 6,338,716 to Hossack et al. describes the use of an ultrasonic transducer probe with a position and orientation sensor. It too suffers from the above limitations.

There is thus a widely recognized need for, and it would be highly advantageous to have a medical imaging system devoid of the above limitations.

### SUMMARY OF THE INVENTION

According to one aspect of the present invention there is provided apparatus for precision location of a tool within an obscured region, the apparatus comprising:

a planar scanning unit for scanning planes within the obscured region using an imaging scan, and

a locator, associated with the tool for determining a location of the tool, and for selecting a plane including the tool location. In a preferred embodiment the locator is operatively associated with the scanner to automatically direct the scanner to the selected plane. However as an alternative the scanner may be handheld. The locator may simply issue a signal, telling the holder of the scanner whether he is scanning the correct plane.

Preferably, the planar scanning unit is a three-dimensional planar scanning unit configured to build a three-dimensional image by combining scans from a plurality of scan planes, and wherein the selecting comprises selecting planes in different orientations that include the tool location.

Alternatively, the planar scanning unit is a three-dimensional planar scanning unit configured to build a three-dimensional image by combining scans from a plurality of scan planes, and selecting comprises selecting from the plurality those planes including the tool location.

Preferably, the locator is user interactive to allow a user to define a feature within a scan, thereby to obtain co-ordinates of the feature to control the scanning unit to scan the feature.

Preferably, the locator is an image processor, associated with the scanning unit, and configured to process results of the scan therefrom to recognize the tool within the scan, thereby to determine the location.

Preferably, the image processor is further operable to recognize and follow predetermined tissue features shown in the scan.

Preferably, the image processor is user interactive to allow a user to define a feature within a scan for following by the image processor, thereby to control the scanning unit to scan the feature.

Preferably, the tool comprises a fluid route for introducing a fluid into the tool.

Preferably, the fluid route comprises an inlet, a reservoir region located about an operating end of the tool and an outlet.

Preferably, the fluid route is filled with bubbled fluid.

Additionally or alternatively, the fluid route is filled with a contrast agent.

In one preferred embodiment, the tool is coated with a substance selected to provide contrast in the scan.

Preferably, the substance is a contrast agent.

Additionally or alternatively, the substance is an ultrasound reflection agent.

Preferably, a tip of the tool is at least coated with a substance selected to provide contrast in the scan, thereby to provide precise location of the tip. Preferably, the substance is a contrast agent. Alternatively, the substance is an ultrasound reflection agent.

In another embodiment, the tool comprises an active ultrasound generator.

The planar scanning unit may be an ultrasonic scanning unit.

The ultrasonic scanning unit may be a 3-dimensional ultrasonic scanning unit configured for planar scanning over a plurality of scan planes and the locator may be configured to direct the 3-dimensional ultrasonic scanning unit so as to include the tool location within regions to be scanned of at least two of the scan planes.

The surgical tool may itself comprise a beacon, and the locator may comprise a corresponding sensor configured to locate the tool by sensing the beacon.

The beacon may comprise an electromagnetic wave generator.

The electromagnetic wave generator may be for example an RF generator, a Pico wave generator, a microwave generator, an infra-red wave generator, a light generator, or an x-ray generator.

The beacon may comprise an ultrasound generator, or even a shockwave generator.

In a preferred embodiment, the beacon is arranged with at least one other beacon to provide a multi-transmitter remote positioning system and the sensor comprises a receiver for contrasting signals from the remote positioning system to determine co-ordinates relative thereto.

Preferably, at least one of the beacons comprises an electromagnetic wave generator.

The electromagnetic wave generator may be for example any of an RF generator, a Pico wave generator, a microwave generator, an infra-red wave generator, a light generator, and an x-ray generator.

At least one of the beacons may comprise an ultrasound generator. Additionally or alternatively, at least one of the beacons comprises a shockwave generator.

In another preferred embodiment, the locator comprises:

a multi-transmitter remote positioning system, and

a receiver for contrasting signals from the remote positioning system to determine coordinates relative thereto.

Preferably, the receiver is located on the tool. Alternatively, at least one transmitter of the multi-transmitter remote positioning system is located on the tool.

The multi-transmitter remote positioning system may comprise at least one electromagnetic wave generator.

As in the previous embodiment, the electromagnetic wave generator may for example comprise an RF generator, a Pico wave generator, a microwave generator, an infra-red wave generator, a light generator, or an x-ray generator.

The multi-transmitter remote positioning system may comprise at least one ultrasound generator.

Preferably, the multi-transmitter remote positioning system comprises a shockwave generator.

In a preferred embodiment, the tool comprises a 3-dimensional accelerometer array and the locator comprises processing functionality for determining a 3-dimensional location from the output of the accelerometer array.

In another preferred embodiment, the tool is attached to a robot arm for movement within the obscured region, and the locator comprises functionality for tracing positioning of the robot arm. The arm is typically segmented and the locator comprises position detectors at each segmentation so that it can accurately find the tool tip position.

The obscured region is typically an intra-cavity region of a human or animal body.

In the preferred embodiments, the locator dynamically updates the tool tip position, thereby to provide dynamic following of the tool. Typically, the locator updates the location following movement of the scanning unit.

According to a second aspect of the present invention there is provided apparatus for precision location of a tool within an obscured region, the apparatus comprising:

a planar scanning unit for scanning planes within the obscured region using an imaging scan, and

a locator, associated with the scanning unit, for determining a location of the tool, and for controlling the scanning unit to follow the tool.

Preferably, the locator is arranged to determine a location of a tip of the tool.

According to a third aspect of the present invention there is provided a method of imaging a tool in an intra-body space comprising: scanning the intra-body space, locating the tool, and using the locating to control the scanning to follow the tool.

Preferably, scanning comprises planar scanning and controlling comprises selecting a scan plane to include at least a tip of the tool within a region to be scanned.

Preferably, scanning is three-dimensional planar scanning comprising scanning using a plurality of planar scans, and controlling comprises including at least a tip of the tool within regions to be scanned of at least two of the scan planes.

Additionally or alternatively, scanning is three-dimensional planar scanning comprising scanning a plurality of planes within the volume and controlling comprises selecting from the plurality, scans including the tip.

Additionally or alternatively, scanning is three-dimensional planar scanning, and controlling comprises selecting a plurality of scan planes in different orientations meeting at the location, for scanning.

Preferably, the process of locating includes providing the tip with recognizability within a scan, for example for ultrasound, one way of providing recognizability is to introduce a bubbled fluid into the tip.

Whatever form of recognizability if provided, the tip can then be identified using image processing, sensitized to the specific form of recognizability.

Other forms of introducing recognizability include using ultrasound contrast agent, ultrasound reflection material, and an active ultrasound signal producer.

Again, recognizability may comprise a signal beacon mounted on the tool, and corresponding locating comprises sensing a signal from the signal beacon.

Locating may comprise providing multi-position interference signaling and at the tool receiving the signals and calculating co-ordinates relative thereto.

In one embodiment locating comprises measuring accelerations in respective dimensions at the tool and calculating a location therefrom.

In one embodiment, the tool is located on a movable robot arm and locating comprises tracking movement of the robot arm.

The method is preferably used for locating the tool within an obscured body region, and scanning comprises scanning at least partly from outside the obscured body region using a type of scan transparent to body tissues.

One preferred embodiment utilizes user interaction to locate a feature in the scan, finding three-dimensional co-ordinates of the feature and then controls the scanning to scan the feature during subsequent movement.

An alternative embodiment utilizes user interaction to locate a feature in the scan, and then uses image processing to follow the feature and control the scanning to scan the feature.

According to a fourth aspect of the present invention there is provided a method of imaging a tool in an intra-body space comprising: determining a location of the tool in three dimensions, and using the location to control planar scanning to follow the tool by including the tool in at least one plane being scanned.

Preferably, the controlling comprises selecting the at least one plane being scanned to include a tip of the tool within an area of the plane being scanned.

Preferably, the scanning is three-dimensional planar scanning comprising scanning using a plurality of planar scans, and the controlling comprises including at least a tip of the tool within regions to be scanned of at least two of the planes being scanned.

Preferably, scanning is three-dimensional planar scanning comprising scanning a plurality of planes within the volume and controlling comprises selecting from the plurality, scans including the tip.

Additionally or alternatively, scanning is three-dimensional planar scanning, and controlling comprises selecting a plurality of planes to be scanned in different orientations meeting at the location, for scanning.

Preferably, scanning comprises providing the tip with recognizability within a scan.

Preferably, locating is achieved by applying image processing, sensitized to the recognizability, to the scanning, to recognize the tip.

Preferably, recognizability comprises applying ultrasound contrast agent, ultrasound reflection material, or an active ultrasound signal producer to the tool tip to assist with automatic recognition.

In a preferred embodiment, mounting a signal beacon on the tool may confer recognizability, and locating comprises sensing a signal from the signal beacon.

In a preferred embodiment, determining a location comprises providing multi-position interference signaling and at the tool receiving the signals and calculating coordinates relative thereto.

Additionally or alternatively, determining a location comprises measuring accelerations in respective dimensions at the tool and calculating a location therefrom.

Additionally or alternatively, the tool is located on a movable robot arm and the determining a location comprises tracking movement of the robot arm.

Typically the tool is located within an obscured body region and scanning is preformed at least partly from outside the obscured body region using a type of scan transparent to body tissues.

The method may utilize user interaction to locate a feature in the scan, finding three-dimensional co-ordinates of the feature, and then control the scanning to follow and continue to scan the feature.

Additionally or alternatively, the method may utilize user interaction to locate a feature in the scan, and then use image processing to follow the feature and control the scanning to scan the feature.

According to a fifth aspect of the present invention there is provided a surgical tool for use with ultrasound imaging, the tool comprising a a region of high contrast to ultrasound about a tip of the tool.

Preferably, the region of high contrast comprises a fluid reservoir connected between a fluid inlet and a fluid outlet, into which a bubbled fluid is injectable.

According to a sixth aspect of the present invention there is provided a surgical tool for use with ultrasound imaging, the tool comprising a a region of automatically variable contrast to ultrasound about a tip of the tool. Preferably, the region of automatically variable contrast comprises a fluid reservoir connected between a fluid inlet and a fluid outlet, into which a bubbled fluid can be injected.

Unless otherwise defined, all technical and scientific terms used herein have the same meaning as commonly understood by one of ordinary skill in the art to which this invention belongs. The materials, methods, and examples provided herein are illustrative only and not intended to be limiting.

According to actual instrumentation and equipment of preferred embodiments of the method and system of the present invention, certain steps such as scanning control and image processing may be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described. by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a generalized schematic diagram showing a tool location apparatus according to a first preferred embodiment of the present invention;

FIG. 2A is a simplified schematic diagram showing a series of parallel scanning planes;

FIG. 2B is a simplified schematic diagram showing the series of parallel scanning planes as produced using a hand-held scanner;

FIG. 2C is a simplified schematic diagram showing the series of parallel scanning planes as produced using a mechanically operated scanner;

FIG. 3A is a simplified schematic diagram showing two non-parallel scanning planes;

FIG. 3B is a simplified diagram showing a series of non-parallel scanning planes produced by rotation of a standard scanner;

FIG. 3C is a simplified diagram showing a series of non-parallel scanning planes produced by a rotary scanner;

FIG. 4 is a simplified schematic diagram showing an image of a body cavity with a surgical tool inserted therein, from which the location of the tool may be determined by image processing, operative in accordance with a preferred embodiment of the present invention;

FIG. 5 is a simplified schematic diagram showing a tool in a body cavity and having a beacon to assist with location, operative in accordance with a preferred embodiment of the present invention;

FIG. 6 is a simplified schematic diagram showing a tool in a body cavity having a receiver for receiving signals from a multi-transmitter positioning system, operative in accordance with a preferred embodiment of the present invention;

FIG. 7 is a simplified diagram showing an alternative embodiment, operative in accordance with a preferred embodiment of the present invention, of the tool of FIG. 6 in which one of the multi-transmitter positioning system transmitters is located on the tool and the receiver is located elsewhere;

FIG. 8 is a simplified diagram showing a tool in a body cavity having an array of accelerometers for position determination, operative in accordance with a preferred embodiment of the present invention;

FIG. 9 is a simplified schematic diagram showing a tool in a body cavity held by a robot arm and wherein the position of the tool is determined by measuring angles at the joints of the robot arm, operative in accordance with a preferred embodiment of the present invention;

FIG. 10 is a simplified flow chart showing a method of scanning a body cavity and using image processing to determine a tool location, operative in accordance with a preferred embodiment of the present invention;

FIG. 11 is a simplified flow chart showing a method of scanning a body cavity using a tool location scheme separate from imaging of the scan, operative in accordance with a preferred embodiment of the present invention;

FIG. 12 is a simplified flow chart showing a method of 3-D scanning of a body cavity and using image processing to determine a tool location, operative in accordance with a preferred embodiment of the present invention; FIG. 13 is a simplified flow chart showing a method of 3-D scanning of a body cavity using a tool location separate from imaging of the scan, operative in accordance with a preferred embodiment of the present invention;

FIG. 14 is a simplified diagram showing a surgical tool with a contrast intensifier for location by an ultrasound scanner according to a preferred embodiment of the present invention;

FIG. 15 is a simplified diagram showing another surgical tool having a bubble canal contrast intensifier according to another preferred embodiment of the present invention;

FIG. 16 is a simplified diagram showing the tool of FIG. 15 in greater detail;

FIG. 17 is a simplified diagram showing the tool of FIG. 15 at a different angle; and

FIG. 18 is a simplified diagram showing a scanner for obtaining scan planes according to a preferred embodiment of the present invention.

### DESCRIPTION OF THE PREFERRED EMBODIMENTS

The present embodiments describe a method and apparatus for carrying out selected planar image scanning, to support and improve scanning orientation for surgery using a surgical tool located within target tissue. The embodiments determine the position of the tool or tool tip and ensure that the selected and presented image scanning is carried out in a plane that includes the tool or tool tip. In one embodiment, the actual scanning co-ordinates are used in combination with image processing of the scan in order to locate the tool.

The present embodiments may for example support real-time sonography using multi planar scanning techniques, based on a three dimensional dataset. The embodiments may be useful for example in providing automatic guidance during intrauterine surgical procedures. The embodiments may use real-time tracking and automated identification of a surgical tool, and provide the surgeon with real-time visualization of the operation target as well as the applied surgical tool within the treatment area, for example a uterine cavity.

The embodiments may diagnose or treat uterine abnormalities , or may for example guide the needle tip during amniocentesis more effectively than in the prior art by providing full tracking of the tool in use, and other areas of interest, during treatment.

The invention allows procedures to be performed in the clinic by any gynecologist or surgeon with general expertise in ultrasonography.

The embodiments eliminate the need for blind surgical procedures under general anesthesia, and thereby reduce complications and improve accuracy. Reduction in complications leads to lower overall cost, and the embodiments specifically provide a solution to many patients for whom blind surgical procedures are considered too risky.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

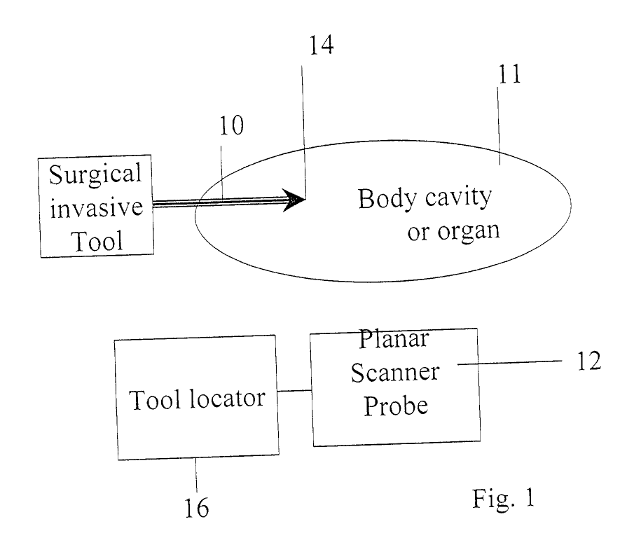

Referring now to the drawings, FIG. 1 is a simplified diagram showing a tool tip location apparatus operative in accordance with a first embodiment of the present invention. A tool 10 is located within an obscured region such as an internal body cavity or organ 11 for the purpose of carrying out an operation. A planar scanning unit 12 is located anywhere and scans the targeted area. The scanning unit 12 scans two-dimensional planes within the cavity or organ, and a three-dimensional scan image may be built up by computing the planes together. In order to carry out the operation the surgeon requires detailed information of the location of a tip 14 of the tool and, as discussed above in the background, it is difficult for a surgeon to keep the scan accurately focused on the tool tip 14, that is to say it is difficult to ensure that the tool tip always falls within a plane being scanned. In addition it is tricky to simultaneously ensure that the area being treated is reasonably well imaged. It is further difficult to coordinate simultaneously both the scanner and the tool together on the same plane. There is thus provided a locator 16, which is able to determine the location of the tool in three dimensions within the cavity or within the organ. The locator 16 may use any suitable method of locating the tool, and several examples are given below. The locator 16 is integrated within the scanning unit 12 and uses the tool tip location (or other selected features) to improve the scanning results.

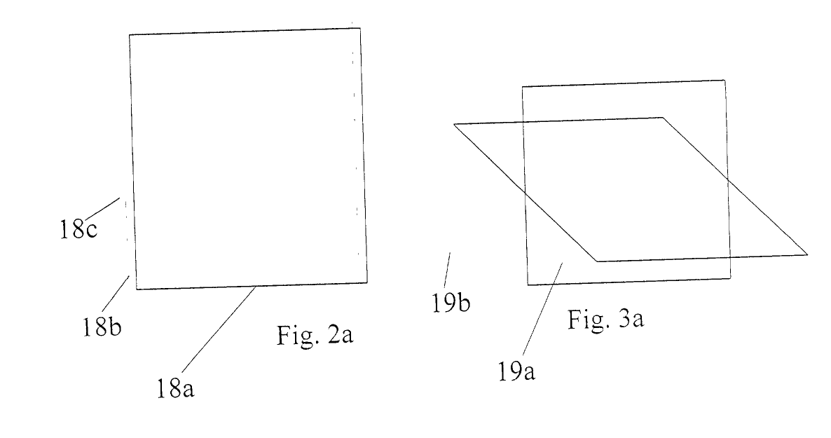

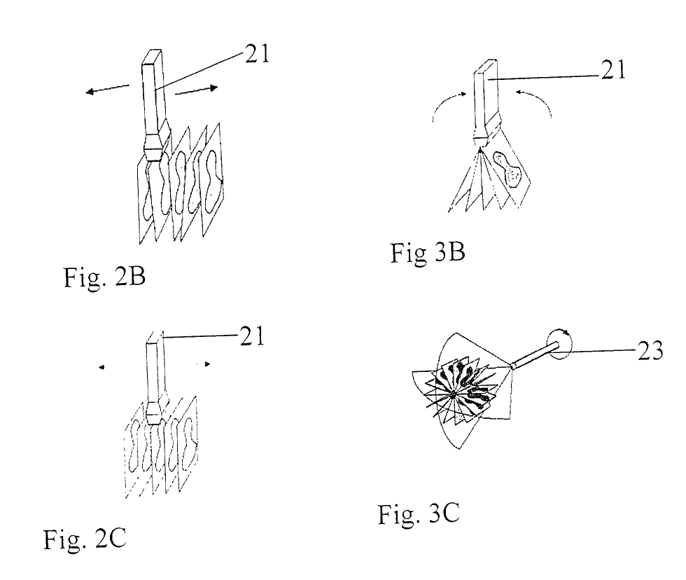

Improvement of the scanning results may be achieved in any one of a number of ways. Firstly, with reference to FIG. 2A, showing a series of parallel original (not computed )planes 18a, 18b, and 18c, the scanner may select a plane ( the closest one) that includes the tool tip or other selected regions, from the series of planes already scanned, for emphasis. Thus a selected plane found to have the tool tip included therein may provide the surgeon with more detail information regarding the treated area and the location of the selected tool in that specific area. As will be explained below, in addition to the tool tip, in some of the embodiments it is possible to define and lock on to tissue features as well, so that several planes could be emphasized in a scan, one for the tool and one each for a series of user identified features.

Secondly, with reference to FIG. 3A, the scanner may select a series of nonparallel planes (or computed planes ) 19a, 19b, that meet at the location of the tool tip, and then control the scanner to scan the series of planes. Thus, in this second method a spherical volume is acquired, in a series of fan-shaped sections of the sphere, with the tool tip at the center. In fact the figure shows only two such planes, and in a preferred embodiment of the present invention one image plane is selected to show the line, that is the longitudinal axis, of the surgical tool, and the other image plane is selected to show the tool tip as a point where it contacts the surrounding tissue. Planes may also be selected to show relationships between different tools or different features. If several planes are used then image processing techniques known to the skilled person can be used to fuse data from the planes to form a 3D image.

FIG. 2B shows how a series of planes may be gathered by movement of a scanner 21. In FIG. 2B the scanner is moved by hand and thus the orientation and the spacings of the scan are irregular. In FIG. 2C the scanner is mechanically controlled, freeing the surgeon or his assistant from having to orient the scanner. In the mechanical version, regular spacings are achieved, but at the cost of control over the scanner. The present embodiments, by inputting the location of the tool to the scanner to set scan positions, provide the advantages of hand and machine scanning together.

FIG. 3B shows how scanner 21 can be rotated to give a series of non-parallel scan planes as in FIG. 3A. FIG. 3C shows a rotary scanner 23 which may be rotated automatically to provide a series of non-parallel planes describing a spherical volume.

A plane that is selected may thus be the plane that includes the tip of the surgical instrument being used. Now the surgeon may be using a three-dimensional model for viewing during the surgery, and in one embodiment, the surgeon is able to project the model onto the patient himself. In another embodiment the three dimensional view may be integrated with the surgeon's view. Such embodiments are useful for intraoperative anatomy exploration, orientation and manipulation, and may also be used in telesurgery systems, where the surgeon controlling the operation is remote from the patient. 3-D image modeling is widely used in neurosurgery in which a 3-D imagebased model of the brain may be presented to the surgeon in a realistic form through the use of stereoscopic displays. Using the display the surgeon is able to more accurately localize the target and plan the trajectory of approach while avoiding sensitive structures.

Fusion techniques can be used and the real world of the operating room (via stereoscopic video images) and the digital MR image of the patient's operation target area, such as uterus or brain may be merged in order to allow the surgeon to visualize the target prior to surgery. A similar procedure using a laser scanner to image the cortical surface may also be used to track the shift of the brain during open craniotomies.

The system of the present embodiments may be used in combination with visual or other forms of feedback. Feedback of the kind used in surgery is well-known if not currently greatly utilized.

Medical images are visual representations of solid structures with different mechanical, textural and functional properties. Even when cursor probes are provided to interrogate the volume, the cursor is generally allowed to roam freely through the volume and there is no feedback to the operator to prevent him from moving beyond organ or tissue, or at least to sensitize him to the fact that such boundaries exist. On the other hand, a clinician who examines an organ, either in-vivo or in-vitro, relies as much on tactile feedback as he does on its appearance.

Until recently, work in the area of providing tactile feedback to enhance the interpretation of medical images has been limited by the speed of generally available computational facilities. Nevertheless, some recent preliminary studies have demonstrated the efficacy of combining 3-D imaging with hepatic interfaces in these circumstances. The use of such an interface in the context of IGNS is considered, particularly to facilitate the positioning of modeled lesions, as well as navigating within the brain with stimulation or lesioning probes, and endoscopes. In each case, tactile feedback, in the form of forces or vibrations, are relayed to the surgeon via a computer-linked, hand-held device. Tactile feedback alerts the surgeon in a natural manner when a proposed lesion position is dangerously close to a critical structure, or when a probe or endoscope is about to enter dangerous territory, for example is about to perforate the ventricular wall.

The addition of tactile feedback to instruments used in image-guided surgery can add an extra layer of confidence to the procedure, by warning or preventing the surgeon from placing a surgical tool in a region considered dangerous, based on analysis of pre-operative 3D medical images

In practising the present invention, the skilled person may come across multimodal registration problems. That is to say major differences in the settings needed and quality of data may arise. Such differences may be due to the type of data to be matched to form the images, the anatomy to be imaged, specific clinical requirements of the particular procedure being supported, and the signal being provided by the surgical tool. Also, differences in registration success may depend on what feature is being looked at. Some features may be easier to locate and follow than others. The user wishes to achieve accurate, steady and repeatable 3D positioning.

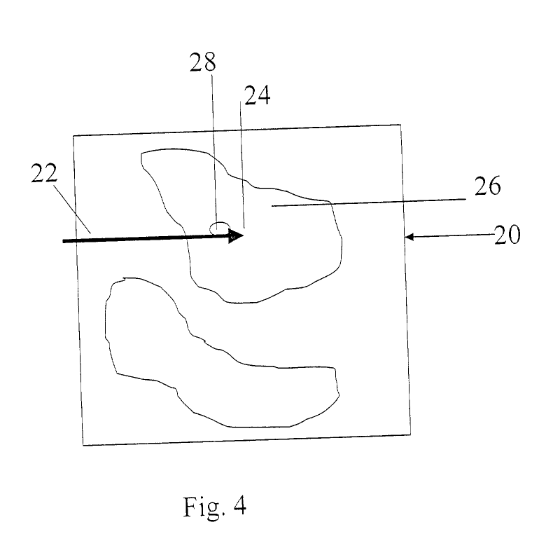

Reference is now made to FIG. 4, which is a simplified diagram showing a scan image 20 having a tool 22 with a tool tip 24 located amongst some body tissue 26 being the subject of the operation. The locator is an image processor which is configured to process the scan image to recognize the tool. Recognition of the tool may be achieved in a number of ways. For example the tool tip 24 may be made of, or at least be coated with, a substance selected to provide a contrast in the scan over the surrounding tissue 26. Thus the image processor simply looks for the region of high contrast and takes that as the location of the tool tip. For ultrasound scanning there are commercially available contrast agents that can be used to coat the tool or tool tip. As an alternative a reflection contrast agent may be used, again to coat the tool or tool tip. For other forms of scanning there are equivalent substances.

The above-described agents all provide passive tool tip location. It is also possible to provide active tool location, and the tool may be fitted with an active ultrasound generator, for example a high frequency magnet-based vibrator type transmitter 28. Upon activation of the transmitter, the tool tip emits an specific ultrasound signal, which may be picked up by the current scan, and processed by the image processor in the same way as the high contrast point of the passive location embodiment.

An advantage of the embodiments described with respect to FIG. 4 are that, since ultrasound is used as the tool location medium, via the scanned images themselves, the determined location of the tool tip is automatically coordinated with the scan. When the tool tip, or any other requested site, is found by the image processor in a given scanned plane, then if the scanned plane is the x, y, plane, the scanner is able to provide the z co-ordinate, and the image processor provides the x and y co-ordinates.

In addition to identifying and locating the tool, the locator is also able to identify and locate a feature in the targeted tissue. The operator may recognize a tissue feature of interest in the scan and flag it as a point of interest. Flagging may be carried using a mouse and cursor or using a touch screen or by any other suitable method. The locator is able to find the z-axis of the scan plane being considered, and the user selection provides x and y co-ordinates. Subsequently, movement of the feature may be tracked by image processing or the system may simply assume that the body is at rest and continue to image the same co-ordinates. Active tracking of the feature of interest is advantageous in that it compensates for involuntary body movements including pulse and breathing related movements, which can be significant in relation to the scale of features involved in some types of operation.

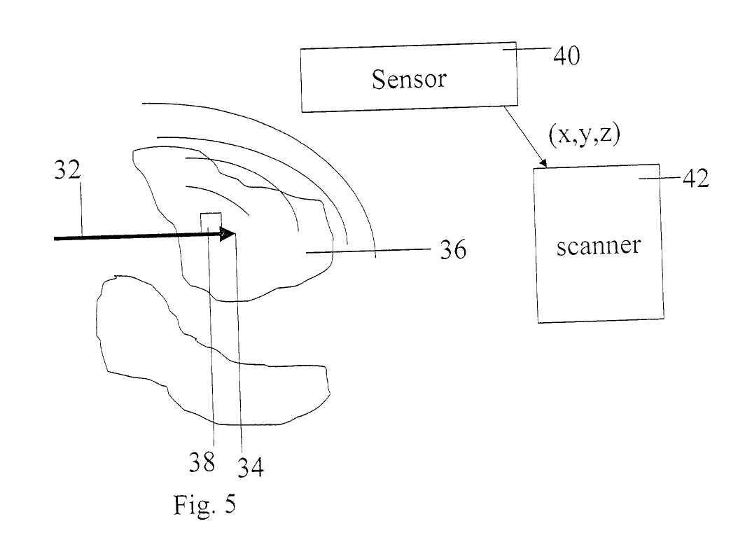

Reference is now made to FIG. 5, which is a simplified diagram showing a further embodiment of a tool location apparatus according to a further preferred embodiment of the present invention in which image scanning and tool location are carried out using separate media. Tool 32, has a tool tip 34 which is located against body tissue 36 on which an operation is to be performed. Located in association with the tool tip 34 is a beacon 38, which emits a signal allowing it to be located in three dimensions. Sensing apparatus 40, senses the signal and determines the co-ordinates (x,y,z) of the tool, which co-ordinates are then used by the scanning unit 42 to scan in the region of the tool tip. The signal used by the beacon may be any signal that is able to exit the cavity and may include radio, x-ray, and ultrasound signals. If an ultrasound signal is used, however, it is generally easier to use the ultrasound image scanner for detection as described in respect of FIG. 4 above, rather than to install a separate location sensor as per the present embodiment.

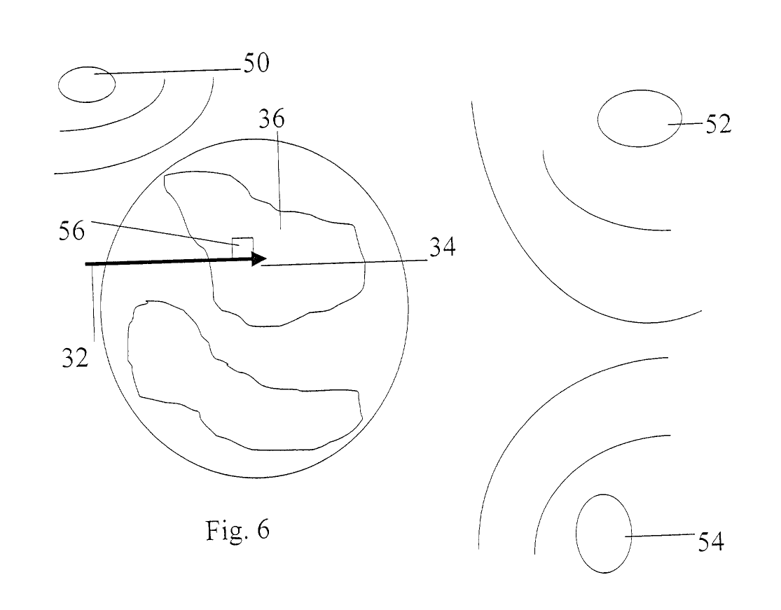

Reference is now made to FIG. 6, which is a simplified alternative embodiment for providing a location of a tool tip according to the present invention. Parts that are the same as those in previous figures are given the same reference numerals and are not referred to again except as necessary for an understanding of the present embodiment. The locator comprises a multi-transmitter remote positioning system, similar to the global positioning system except on a vastly smaller scale. The positioning system comprises a series of transmitters 50, 52, 54, each emitting a signal. The tool 32 comprises a receiver 56 which receives the signals from each of the transmitters. The received signals are compared and a position is determined relative to the transmitters. The determined position is then relayed to the scanning unit as before.

The positioning system may make use of any kind of electromagnetic waves including RF, magnetism, microwave, infra-red, light, ultra-violet, and x-ray. Light may involve following of LEDs located on the tool, or image processing to follow the tool or other known object. Magnetism may involve the placing of a magnet on the tool and sensing changes in magnetic field as a consequence of moving the tool. If the tool is being used in an intra-body cavity or other obscured location then the skilled person may take care to ensure that the positioning system uses a part of the spectrum that is able to penetrate the obscuring material. Aside from electromagnetic waves the positioning system may use ultrasound, shock waves or any other suitable kind of wave.

With further regard to the use of magnetism, such magnet-based technology, known as electromagnetic (EM) surgical navigation, is transparent to the user, and transparent to the procedure type. Line-of-sight restrictions are eliminated, as well as the need for any change in surgical flow or technique. An algorithm known as Magneticlntelligence™, of General Electric Corporation, automatically detects and compensates for metal in the field, improving accuracy.

The use of electromagnetism together with planar imaging in accordance with the above-described embodiments provides three-dimensional visualization of a patient's anatomy, and the ability to track the position and orientation of instrumentation during surgery.

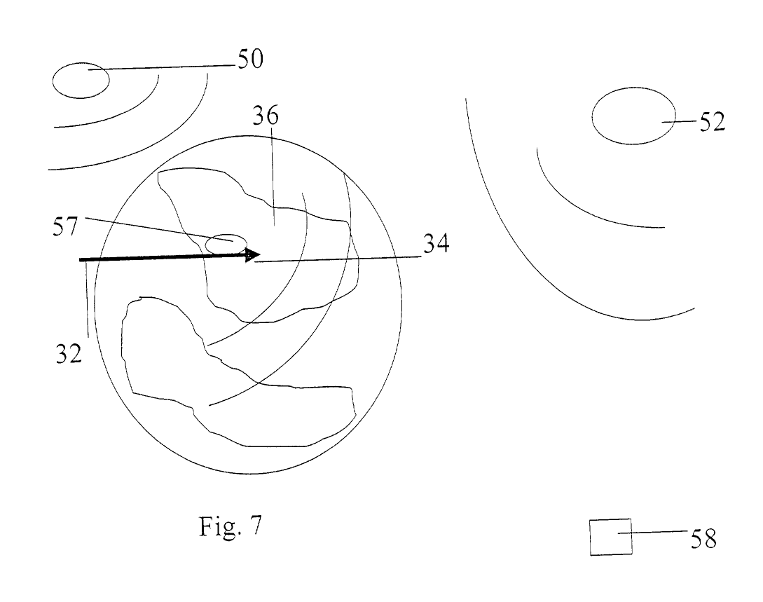

Reference is now made to FIG. 7, which is a simplified diagram showing a variation of the embodiment of FIG. 6. Parts that are the same as in FIG. 6 are given the same reference numerals and are not described again except to the extent necessary for an understanding of the present variation. The multi-transmitter positioning system includes a transmitter 57 located in the region of the tool tip. A receiver 58 is located away from the tool. The receiver 58 receives signals from each of the transmitters and uses phase differences and other contrasts between the signals to determine the position of the tool tip in three dimensions. That is to say, instead of providing a receiver on the tool, a transmitter is provided on the tool, and a receiver compares between signals from the moving tool tip and from stationary transmitters. An advantage of the variation of FIG. 7 is that the tool does not have to have access to processing power. By contrast the receiver on the tool of FIG. 6 must be able to compare received signals or transfer them to another location able to carry out a comparison without distorting phase information.

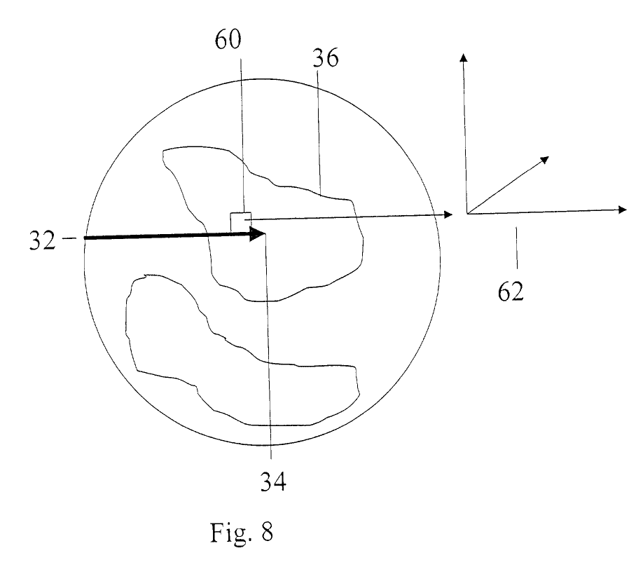

Reference is now made to FIG. 8, which is a simplified schematic diagram showing a further preferred embodiment for obtaining a tool location, operative in accordance with the present invention. Parts that are the same as those in previous figures are given the same reference numerals and are not referred to again except as necessary for an understanding of the present embodiment. In the embodiment of FIG. 8, tool 32 comprises an accelerometer array. The array comprises three accelerometers placed mutually perpendicularly to each other, as shown by arrow arrangement 62, so as to record acceleration in three dimensions. The tool begins each operation or part thereof at a predetermined starting point, and then tracking of the acceleration is subsequently sufficient to provide accurate positioning. The embodiment of FIG. 8 is advantageous in that it does not require any kind of radiation since signals from the accelerometer can be wired directly to the scanner.

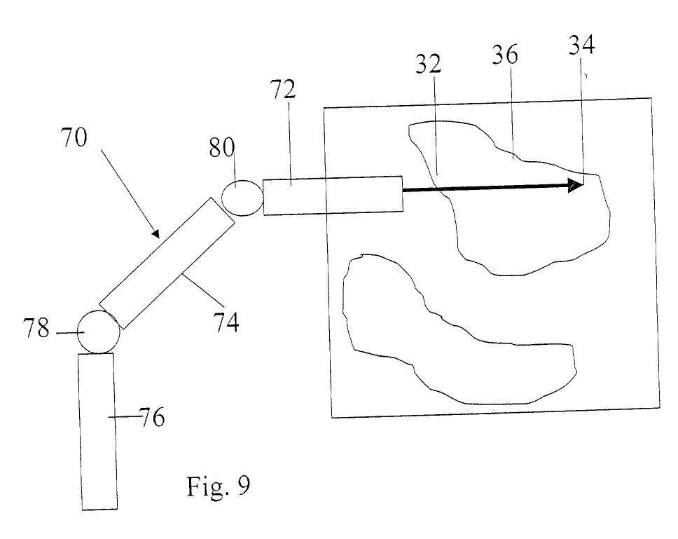

In all of the above embodiments, the tool 32 may be hand held by the surgeon or it may be manipulated by a robot arm. If manipulated by a robot arm then the system can be used in providing remote surgery. Reference is now made to FIG. 9, which is a simplified schematic diagram showing a location system specifically suited to cases in which the tool 32 is mounted on a robot arm 70. The robot arm comprises a series of arm sections 72, 74, 76 with joints 78, 80 in between. At each joint one or more rotation sensor determine the current joint rotation, allowing the position of the end of the arm and thus of the tool to be determined. In general each individual joint can rotate in two dimensions and requires two sensors to measure and fully define the rotation. The sensors may typically be potentiometer-based sensors. An advantage of the embodiment of FIG. 9 is that robot arms comprising such sensors are available as off-the-shelf components, allowing for convenient implementation.

As mentioned above, the system is suitable for following a tool for use in an obscured region. The obscured region may be an intra-body or intra-body cavity region of a human or animal. Scanning systems for scanning intra-body regions are well-known but often because of the planar nature of scanning it can be difficult to keep track of a tool tip being used in an operation. The tip tracking disclosed hereinabove allows the scanning to automatically track the tool tip, thus allowing the surgeon to focus attention on the operation itself.

In a further preferred embodiment of the present invention, the tool locator 16 dynamically updates the tool position as the tool moves, say in the course of carrying out an operation. The updates can then be fed to the scanning system to direct the next scan and thus provide dynamic following of the tool.

Likewise the tool position can be dynamically followed for imaging purposes following movement of the scanner. The surgeon may wish to view the tool and surrounding tissue from different angles or from different distances. Currently, movement of the scanner is tricky because the surgeon has to find a plane that includes the tool tip every time the scanner is moved. With the tool locater system 16 taking over such a plane finding function, scanner repositioning becomes much simpler and the repositioned scanner simply uses the latest co-ordinates of the tool tip.

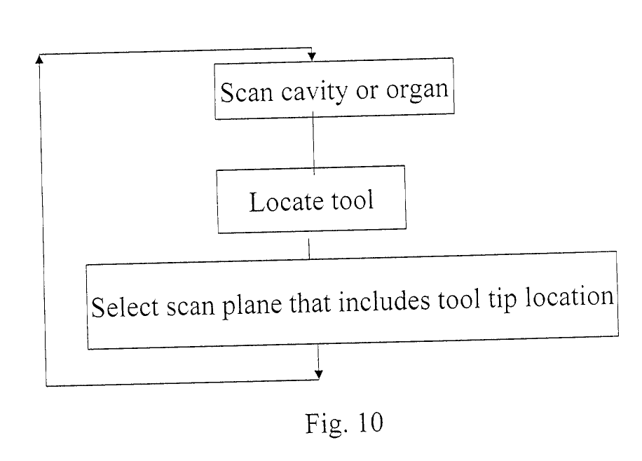

Reference is now made to FIG. 10, which is a simplified diagram showing a method of imaging a tool, for example in an intra-body cavity. The method comprises scanning the intra-body cavity using any suitable scanning method, including ultrasound, magnetic resonance imaging, CT scans and the like. A tool or other foreign body is located within the cavity in three dimensions and then the location is used to direct the scanner to include the tool in its scan. As discussed above, the tool may typically be a surgical tool carrying out an operation. Many scans are planar scans which scan flat planes, and it generally requires significant skill on the part of the surgeon to obtain a scanning plane that actually includes the working tip of his tool. At best the attempt to include the working tip is a significant distraction for the surgeon. In one variation the scan itself is used to identify the tool. Thus in the initial stages the tool tip has to be found manually. Once the tool tip has been found it is identified from the scan by image processing and a location is derived. Then the scanner is controlled to follow the tool tip. As mentioned above, it is possible to enhance recognizability of the tool for the image processor by coating the tool with a contrast agent or a reflection agent. Alternatively an active source on the tool may be used to illuminate the tool in the image.

Upon recognition of the tool or tool tip, the system may select a particular plane including the tool for emphasis. Alternatively it may choose a series of nonparallel planes to scan that each include the tool location.

In a preferred embodiment, the scan is an ultrasound scan and image processing operates on an ultrasound image capture to identify and locate the tool.

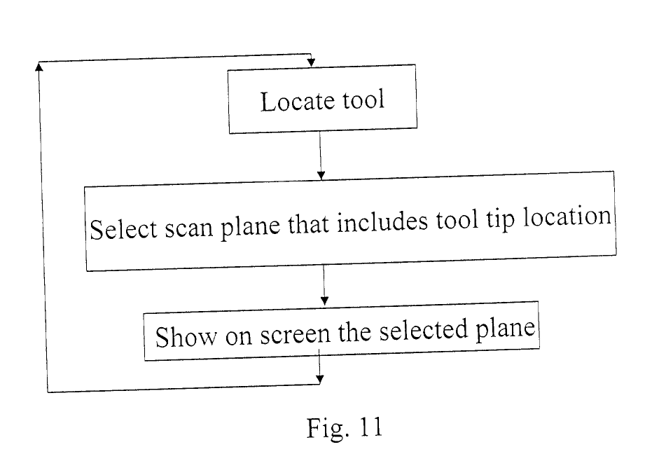

Reference is now made to FIG. 11, which is a simplified flow chart showing a variation of the method of FIG. 10. In the method of FIG. 11, the location and scanning systems are separate in that obtaining the location of the tool in three dimensions is carried out separately from processing of the scan. In such a case the tool location is firstly determined, using any of the methods detailed with respect to FIGS. 5-9 or any other suitable method. Tool location may for example be achieved by receiving transmissions from a beacon located on the tool, at a plurality of locations, and processing the transmission to determine its co-ordinates in three dimensions. As an alternative, discussed with respect to FIG. 6 above, a set of transmitters may be placed around the tool and a receiver placed on the tool. The signals received at the tool receiver may be used to determine the tool's location in three dimensions.

As a further alternative, discussed with respect to FIG. 7 above, one or more transmitters may be located around the tool and a further transmitter on the tool. A receiver may be positioned away from the tool. Location is achieved by comparing signals from the tool and the other transmitters.

A further alternative, discussed with respect to FIG. 8 above, provides for an array of acceleration sensors on the tool to provide acceleration data, from which the current position of the tool can be traced.

Following location of the tool, a scan plane is selected that includes the tool, and then the selected plane is scanned. Thus a scan is produced that automatically includes the tool. Thus the surgeon is provided with a view that shows the tool he is working with. As discussed above, the scan may dynamically follow movements of the tool or alternatively may dynamically compensate for movements of the scanner. for example if the surgeon wishes to scan from a different angle or get closer to his subject.

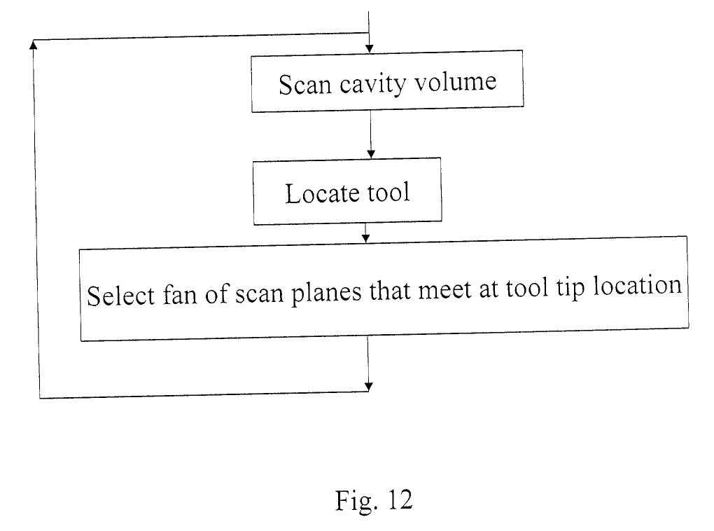

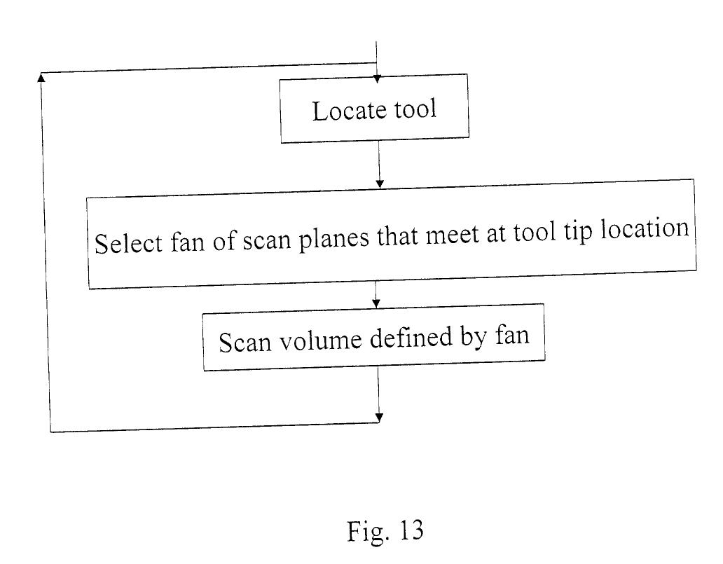

Reference is now made to FIG. 12, which is simplified flow chart showing a variation of the method of FIG. 10 specifically for producing a three-dimensional scan. A volume of interest is scanned and image processing is applied to the scanned planes to locate the tool or tool tip. The ability to locate the tool using image processing may be enhanced by using any of the methods described above, including using a suitable contrast agent or reflection agent. Once the tool has been located then an arrangement of planes is selected to obtain a volume about the tool and to follow the tool.

Likewise it is possible to indicate to the system a region of interest on the image, for example a feature in the tissue. The feature may be indicated by pointing using a cursor or any other suitable method. The locator may simply record the three-dimensional co-ordinates of the feature and continue to scan at those coordinates or it may apply image processing to follow the tissue feature. The latter is useful if the tissue moves, however there is a limit to tissue features that are suitable for following by image processing.

Reference is now made to FIG. 13, which is a simplified flow chart showing a variation of the method of FIG. 11 specifically for forming a three-dimensional scan. The tool location is found as described hereinabove in accordance with any of the methods of FIGS. 5-9, and the location information is used to select planes for scanning that include the tool. Tool location may for example be achieved by receiving transmissions from a beacon located on the tool, at a plurality of locations, and processing the transmission to determine its co-ordinates in three dimensions. As an alternative, discussed with respect to FIG. 6 above, a set of transmitters may be placed around the tool and a receiver placed on the tool. The signals received at the tool receiver may be used to determine the tool's location in three dimensions.

As a further alternative, discussed with respect to FIG. 7 above, one or more transmitters may be located around the tool and a further transmitter on the tool. A receiver may be positioned away from the tool. Location is achieved by comparing signals from the tool and the other transmitters.

A further alternative, discussed with respect to FIG. 8 above, provides for an array of acceleration sensors on the tool to provide acceleration data, from which the current position of the tool can be traced.

Following location of the tool, the selected planes are scanned and an image produced. The process is repeated with the tool location being redetermined. If the tool is found to have moved then new planes are selected and so-on. Thus the system succeeds in dynamically following the progress of the tool through the operation.

In a preferred embodiment of the present invention, image analysis or any of the other methods of tool plane tracing may be carried out in a tracing mode whereas regular scanning is carried out in a scanning mode. The scanner may, at the user's direction pass from one mode to the other. Thus the user may transfer from volume acquiring to tracing mode or vice versa. In tracing mode the scanner may lock on to the tool tip or any other point being indicated and then return to volume acquiring mode proceed to acquire volume whilst following that point so as to constantly include that point in an image plane. Tracing mode may be carried out as discussed above using signal processing or image processing techniques. The embodiment allows computerized movement to replace hand guiding of the scanner. The scanner may nevertheless be handheld, and the locking on feature may allow for compensation for inadvertent hand movements.

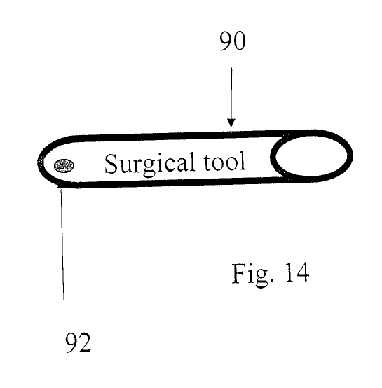

Reference is now made to FIG. 14, which is a simplified diagram showing a tool suitable for use with the embodiments of the present invention. Tool 90 is any kind of invasive tool whose location can be used to control or follow the progress of an operation, and examples include curettes, including the Sims Curette and the Hunter curette, uterine aspiration curettes, both curved and straight, uterine dilators including the Hegar dilator, the Pratt dilator and the Hank dilator, and sponge forceps, including the Foerster, and DeLee ovum forceps.

A point, 92, is selected, preferably as a point that carries out the surgical procedure or the point nearest to the tissue on which the procedure is being carried out, and the point is then marked or signed so that it can be followed. Marking or signing may be carried out using any suitable method, in particular the methods outlined hereinabove.

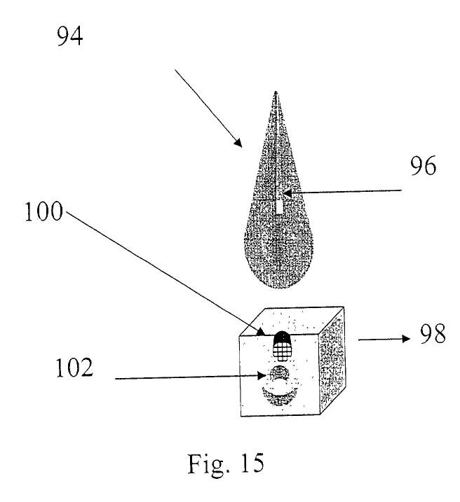

Reference is now made to FIG. 15, which is a simplified diagram showing a surgical tool according to a further preferred embodiment of the present invention. Surgical tool 94 may be any kind of surgical tool. The tool comprises an internal pipe or canal structure 96 that normally contains water. A pump 98 is connected to the tool via connector 100 to pump water into the canal 96. The pump includes a bubble chamber which allows the pump to introduce bubbles into the canal. Bubbles show up brightly with ultrasound and thus the combination of ultrasound and a tool having a bubble canal provides a simple method of allowing the ultrasound to follow the tool. As bubbles can be introduced rapidly, the bubble canal provides a way of achieving high contrast on demand.

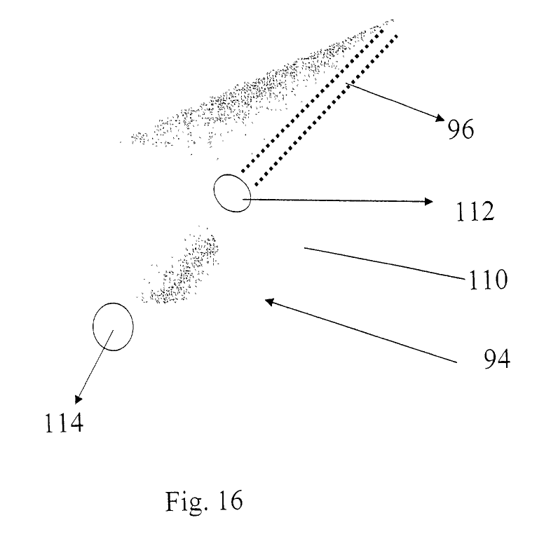

Reference is now made to FIG. 16, which is a simplified diagram showing the tool of FIG. 15 in greater detail. The tool 94 comprises an outer wall 110 into which canal 96 is built. The canal has an outward leg 112 connected to an outlet of the pump connector and a return leg 114 connected to an inlet of the pump.

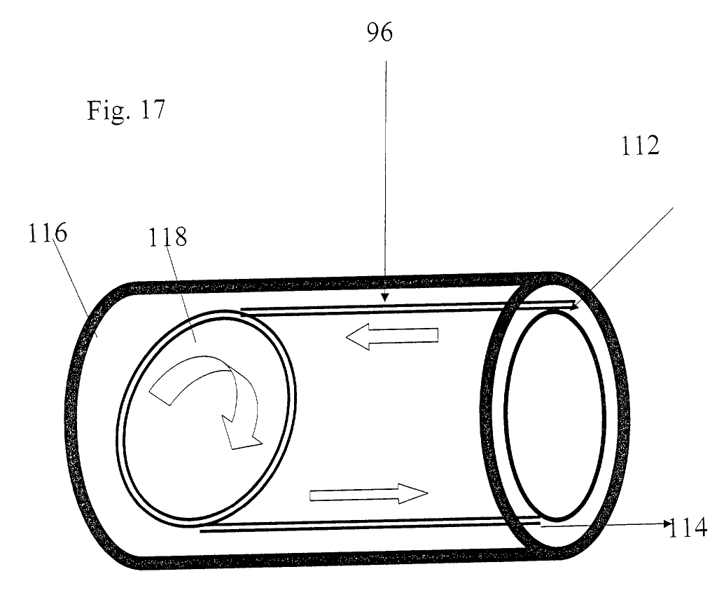

Reference is now made to FIG. 17, which is a simplified diagram showing a further view of the tool of FIG. 16. Parts that are the same as in previous figures are given the same reference numerals and are not described again except to the extent necessary for an understanding of the present figure. At the operative end 116 of the tool 94 the canal forms a reservoir region 118 in order to render itself identifiable to the image processing system referred to above.

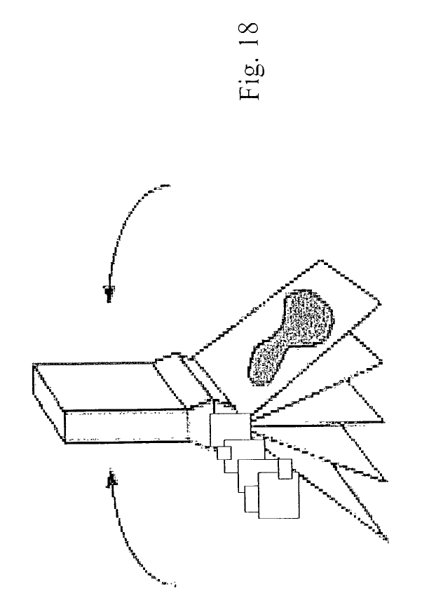

Reference is now made to FIG. 18, which is a simplified diagram showing a scanner obtaining scans of a region of interest. The scanner first scans a series of planes in order to locate a target, such as a tool tip. A plane of interest is identified from the scanned planes using image analysis. Then the scanner locks onto the plane of interest. However the target moves so, whenever the image of the tool grows faint it scans around the current plane of interest to identify a new plane of interest.

The embodiments described above are useful in any kind of activity wherein imaging is needed to see what is happening and interactive feedback is required. Particular applications in the medical field include gynecology and uterine surgery, obstetrics and amniocentesis, chorionic villi sampling, breast biopsy, neurosurgery, orthopedics, maxillofacial, craneofacial and dental surgery, laparoscopic and endoscopic surgery, radiotherapy, and specific procedures in ophthalmology.

It is expected that during the life of this patent many relevant forms of beacon, sensing, and location technology will be developed and the scope of the terms “beacon”, “sensor” and “locator” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. Apparatus for precision location of a tool within an obscured region, the apparatus comprising:
a planar scanning unit for scanning planes within said obscured region using an imaging scan, and
a locator, associated with said tool, for determining a location of said tool, and for selecting a plane including said tool location.

2. The apparatus of claim 1, wherein said locator is additionally operatively associated with said planar scanning unit to automatically direct said planar scanning unit to scan within said selected plane.

3. The apparatus of claim 2, wherein said planar scanning unit is a three-dimensional planar scanning unit configured to build a three-dimensional image by combining scans from a plurality of scan planes, and wherein said selecting comprises selecting planes in different orientations that include said tool location.

4. The apparatus of claim 2, wherein said planar scanning unit is a three-dimensional planar scanning unit configured to build a three-dimensional image by combining scans from a plurality of scan planes, and wherein said selecting comprises selecting from said plurality those planes including said tool location.

5. The apparatus of claim 2, wherein said locator is user interactive to allow a user to define a feature within a scan, thereby to obtain co-ordinates of said feature to control said scanning unit to scan said feature.

6. The apparatus of claim 2, wherein said locator is an image processor, associated with said scanning unit, and configured to process results of said scan therefrom to recognize said tool within said scan, thereby to determine said location.

7. The apparatus of claim 6, wherein said image processor is further operable to recognize and follow predetermined tissue features shown in said scan.

8. The apparatus of claim 6, wherein said image processor is user interactive to allow a user to define a feature within a scan for following by said image processor, thereby to control said scanning unit to scan said feature.

9. The apparatus of claim 6, wherein said tool comprises a fluid route for introducing a fluid into said tool.

10. The apparatus of claim 9, wherein said fluid route comprises an inlet, a reservoir region located about an operating end of said tool and an outlet.

11. The apparatus of claim 10, wherein said fluid route is filled with bubbled fluid.

12. The apparatus of claim 10, wherein said fluid route is filled with a contrast agent.

13. The apparatus of claim 6, wherein said tool is coated with a substance selected to provide contrast in said scan.

14. The apparatus of claim 13, wherein said substance is a contrast agent.

15. The apparatus of claim 13, wherein said substance is an ultrasound reflection agent.

16. The apparatus of claim 6, wherein a tip of said tool is at least coated with a substance selected to provide contrast in said scan, thereby to provide precise location of said tip.

17. The apparatus of claim 16, wherein said substance is a contrast agent.

18. The apparatus of claim 16, wherein said substance is an ultrasound reflection agent.

19. The apparatus of claim 6, wherein said tool comprises an active ultrasound generator.

20. The apparatus of claim 6, wherein said planar scanning unit is an ultrasonic scanning unit.

21. The apparatus of claim 20, wherein said ultrasonic scanning unit is a 3-dimensional ultrasonic scanning unit configured for planar scanning over a plurality of scan planes and wherein said locator is configured to direct said 3-dimensional ultrasonic scanning unit so as to include said tool location within regions to be scanned of at least two of said scan planes.

22. The apparatus of claim 2, wherein said tool comprises a beacon, and said locator comprises a sensor configured to locate said tool by sensing said beacon.

23. The apparatus of claim 22, wherein said beacon comprises an electromagnetic wave generator.

24. The apparatus of claim 23, wherein said electromagnetic wave generator is one of a group comprising an RF generator, a Pico wave generator, a microwave generator, an infra-red wave generator, a light generator, and an x-ray generator.

25. The apparatus of claim 22, wherein said beacon comprises an ultrasound generator.

26. The apparatus of claim 22, wherein said beacon comprises a shockwave generator.

27. The apparatus of claim 22, wherein said beacon is arranged with at least one other beacon to provide a multi-transmitter remote positioning system and wherein said sensor comprises a receiver for contrasting signals from said remote positioning system to determine co-ordinates relative thereto.

28. The apparatus of claim 27, wherein at least one of said beacons comprises an electromagnetic wave generator.

29. The apparatus of claim 27, wherein said electromagnetic wave generator is any one of a group comprising an RF generator, a Pico wave generator, a microwave generator, an infra-red wave generator, a light generator, and an x-ray generator.

30. The apparatus of claim 27, wherein at least one of said beacons comprises an ultrasound generator.

31. The apparatus of claim 27, wherein at least one of said beacons comprises a shockwave generator.

32. The apparatus of claim 2, wherein said locator comprises: a multi-transmitter remote positioning system, and a receiver for contrasting signals from said remote positioning system to determine coordinates relative thereto.

33. The apparatus of claim 32, wherein said receiver is located on said tool.

34. The apparatus of claim 32, wherein at least one transmitter of said multi-transmitter remote positioning system is located on said tool.

35. The apparatus of claim 32, wherein said multi-transmitter remote positioning system comprises at least one electromagnetic wave generator.

36. The apparatus of claim 35, wherein said electromagnetic wave generator is one of a group comprising an RF generator, a Pico wave generator, a microwave generator, an infra-red wave generator, a light generator, and an x-ray generator.

37. The apparatus of claim 32, wherein said multi-transmitter remote positioning system comprises at least one ultrasound generator.

38. The apparatus of claim 32, wherein said multi-transmitter remote positioning system comprises a shockwave generator.

39. The apparatus of claim 2, wherein said tool comprises a 3-dimensional accelerometer array and wherein said locator comprises processing functionality for determining a 3-dimensional location from output of said accelerometer array.

40. The apparatus of claim 2, wherein said tool is attached to a robot arm for movement within said obscured region, and wherein said locator comprises functionality for tracing positioning of said robot arm.

41. The apparatus of claim 40, wherein said arm is segmented and wherein said locator comprises position detectors at each segmentation.

42. The apparatus of claim 2, wherein said obscured region is an intra-cavity region of an animal body.

43. The apparatus of claim 2, wherein said obscured region is an intra-cavity region of a human body.

44. The apparatus of claim 2, wherein said locator is operable to dynamically update said position, thereby to provide dynamic following of said tool.

45. The apparatus of claim 2, wherein said locator is operable to update said location following movement of said scanning unit.

46. Apparatus for precision location of a tool within an obscured region, the apparatus comprising:
a planar scanning unit for scanning planes within said obscured region using an imaging scan, and
a locator, associated with said scanning unit, for determining a location of said tool, and for controlling said scanning unit to follow said tool.

47. The apparatus of claim 46, wherein said locator is arranged to determine a location of a tip of said tool.

48. A method of imaging a tool in an intra-body space comprising:
scanning said intra-body space, locating said tool, and
using said locating to control said scanning to follow said tool.

49. The method of claim 48, wherein said scanning comprises planar scanning and said controlling comprises selecting a scan plane to include at least a tip of said tool within a region to be scanned.

50. The method of claim 49, wherein said scanning is three-dimensional planar scanning comprising scanning using a plurality of planar scans, and said controlling comprises including at least a tip of said tool within regions to be scanned of at least two of said scan planes.

51. The method of claim 49, wherein said scanning is three-dimensional planar scanning comprising scanning a plurality of planes within said volume and said controlling comprises selecting from said plurality, scans including said tip.

52. The method of claim 49, wherein said scanning is three-dimensional planar scanning, and said controlling comprises selecting a plurality of scan planes in different orientations meeting at said location, for scanning.

53. The method of claim 48, wherein said locating comprises providing said tip with recognizability within a scan.

54. The method of claim 53, wherein said providing recognizability comprises introducing a bubbled fluid into said tip.

55. The method of claim 55, further comprising applying image processing, sensitive to said recognizability, to said scanning, to recognize said tip.

56. The method of claim 55, wherein said recognizability comprises one of a group consisting of ultrasound contrast agent, ultrasound reflection material, and an active ultrasound signal producer.

57. The method of claim 48, wherein said recognizability comprises a signal beacon mounted on said tool, and said locating comprises sensing a signal from said signal beacon.

58. The method of claim 48, wherein said locating comprises providing multi-position interference signaling and at said tool receiving said signals and calculating co-ordinates relative thereto.

59. The method of claim 48, wherein said locating comprises measuring accelerations in respective dimensions at said tool and calculating a location therefrom.

60. The method of claim 48, wherein said tool is located on a movable robot arm and said locating comprises tracking movement of said robot arm.

61. The method of claim 48 further comprising locating said tool within an obscured body region and wherein said scanning comprises scanning at least partly from outside said obscured body region using a type of scan transparent to body tissues.

62. The method of claim 48, further comprising using user interaction to locate a feature in said scan, finding three-dimensional co-ordinates of said feature and controlling said scanning to scan said feature.

63. The method of claim 48, further comprising using user interaction to locate a feature in said scan, and using image processing to follow said feature and control said scanning to scan said feature.

64. A method of imaging a tool in an intra-body space comprising:
determining a location of said tool in three dimensions, and
using said location to control planar scanning to follow said tool by including said tool in at least one plane being scanned.

65. The method of claim 64, wherein said controlling comprises selecting said at least one plane being scanned to include a tip of said tool within an area of said plane being scanned.

66. The method of claim 65, wherein said scanning is three-dimensional planar scanning comprising scanning using a plurality of planar scans, and said controlling comprises including at least a tip of said tool within regions to be scanned of at least two of said planes being scanned.

67. The method of claim 65, wherein said scanning is three-dimensional planar scanning comprising scanning a plurality of planes within said volume and said controlling comprises selecting from said plurality, scans including said tip.

68. The method of claim 65, wherein said scanning is three-dimensional planar scanning, and said controlling comprises selecting a plurality of planes to be scanned in different orientations meeting at said location, for scanning.

69. The method of claim 64, further comprising providing said tip with recognizability within a scan.

70. The method of claim 70, further comprising applying image processing, sensitive to said recognizability, to said scanning, to recognize said tip.

71. The method of claim 70, wherein said recognizability comprises one of a group consisting of ultrasound contrast agent, ultrasound reflection material, and an active ultrasound signal producer.

72. The method of claim 64, wherein said recognizability comprises a signal beacon mounted on said tool, and said locating comprises sensing a signal from said signal beacon.

73. The method of claim 64, wherein said determining a location comprises providing multi-position interference signaling and at said tool receiving said signals and calculating co-ordinates relative thereto.

74. The method of claim 64, wherein said determining a location comprises measuring accelerations in respective dimensions at said tool and calculating a location therefrom.

75. The method of claim 64, wherein said tool is located on a movable robot arm and said determining a location comprises tracking movement of said robot arm.

76. The method of claim 64 further comprising locating said tool within an obscured body region and wherein said scanning comprises scanning at least partly from outside said obscured body region using a type of scan transparent to body tissues.

77. The method of claim 64, further comprising using user interaction to locate a feature in said scan, finding three-dimensional co-ordinates of said feature and controlling said scanning to scan said feature.

78. The method of claim 64, further comprising using user interaction to locate a feature in said scan, and using image processing to follow said feature and control said scanning to scan said feature.

79. A surgical tool for use with ultrasound imaging, said tool comprising a a region of high contrast to ultrasound about a tip of said tool.

80. The surgical tool of claim 79, wherein said region of high contrast comprises a fluid reservoir connected between a fluid inlet and a fluid outlet, into which a bubbled fluid is injectable.

81. A surgical tool for use with ultrasound imaging, said tool comprising a region of automatically variable contrast to ultrasound about a tip of said tool.

82. The surgical tool of claim 81, wherein said region of automatically variable contrast comprises a fluid reservoir connected between a fluid inlet and a fluid outlet, into which a bubbled fluid is injectable.
